(19) United States
(12) Patent Application Publication
(10) Pub. No.: US 2021/0307756 A1
(43) Pub. Date: Oct. 7, 2021

(54) TREATMENT SYSTEM AND APPLICATOR

(71) Applicant: OLYMPUS CORPORATION, Tokyo (JP)

(72) Inventors: Kensuke UESAKA, Tokyo (JP); Nobuyuki SUDA, Kamiina-gun (JP); Michitsugu ARIMA, Tokyo (JP)

(73) Assignee: OLYMPUS CORPORATION, Tokyo (JP)

(21) Appl. No.: 17/354,357

(22) Filed: Jun. 22, 2021

Related U.S. Application Data
(63) Continuation of application No. PCT/JP2018/048479, filed on Dec. 28, 2018.

(51) Int. Cl. A61B 17/128; A61B 17/122

(52) U.S. Cl. CPC A61B 17/128; A61B 2017/12004; A61B 17/122

(57) ABSTRACT

A treatment system includes: an implant placed in a body; an insertion portion that is inserted into the body; and a flap having a first end and a second end located at a position closer to a distal end than the first end, the flap extending from the first end to the second end, wherein the second end has a receiving portion through which the implant can enter, and can be arranged at a spaced position, the spaced position being a position more spaced from a central axis of the insertion portion than the first end, and the flaps support the implant by abutting against the implant placed in the body.

### CROSS REFERENCE TO RELATED APPLICATIONS

The present application is a continuation application based on a PCT Patent Application No. PCT/JP2018/048479, filed on Dec. 28, 2018, the entire content of which is hereby incorporated by reference.

### BACKGROUND

### Technical Field

The present invention relates to a treatment system and an applicator.

### Background

Ligation using a clip unit is a known procedure performed using an endoscope. Japanese Patent (Granted) Publication No. 5750620 (hereinafter referred to as Patent Document) discloses a clip unit including a pair of arms. In a state where a pair of arms are pulled by a predetermined amount while the pair of arms sandwich a tissue, the pair of arms are locked with the tissue strongly tightened.

In a state where the area to be ligated is wide, a plurality of clip units may be placed in the tissue. For example, when a new clip unit is placed near a clip unit that has already been placed, the clip unit that has already been placed may interfere with the treatment.

The present invention provides a treatment system capable of performing treatment while suppressing interference of surrounding structures.

A treatment system includes: an implant placed in a body; an insertion portion that is inserted into the body; and a flap having a first end and a second end located at a position closer to a distal end than the first end, the flap extending from the first end to the second end, wherein the second end has a receiving portion through which the implant can enter, and cats be arranged at a spaced position, the spaced position being a position more spaced from a central axis of the insertion portion than the first end, and the flap support the implant by abutting against the implant placed in the body.

The treatment system may further include: an operation wire separably connected to at least one of the first arm and the second arm; and a tubular holding tube into which at least one of the first arm and the second arm is inserted.

The first end may be fixed to the insertion portion al a position closer to a distal end of the first arm and a distal end of the second arm, and the second end may be located closer to the distal end of the first arm and the distal end of the second arm.

The first end may be fixed to the insertion portion at a a more proximal end side position than a distal end of the first arm and a distal end of the second arm, the second end may be located closer a proximal end side than the distal end of the first arm and the distal end of the second arm, and in a state where the second end is arranged at the spaced position, a length from the distal end of the first arm to the second end in a direction along the central axis and a length from the distal end of the second arm to the second end in the direction along the central axis may be shorter than a maximum length of the implant.

The first end may be configured to be movable in a direction along the central axis with respect to the first arm and the second arm.

An applicator to which a clip unit having a first arm and a second arm is separably connected includes: an insertion portion configured to be inserted into a body; and a plurality of flaps extending from a first end to a second end arranged on a more distal end side than the first end, wherein the second end is arranged at a spaced position, the spaced position being a position more spaced from a central axis of the insertion portion than the first end, and in a state where the clip unit and the applicator are connected, the spaced position of the second end of at least one of the flaps is located on a second plane that intersects the first plane including the first arm, the second arm, and the central axis of the insertion portion.

The second end may be capable of being moved to a closer position, the closer position being a position closer to the central axis than the spaced position.

The applicator may further include: an outer sheath having a tubular shape into which the insertion portion and the flap can be inserted, wherein the second end may be urged in a direction away from the central axis, and the second end may move to the spaced position by moving from an inside of the outer sheath to an outside.

The first end may be configured to be movable in a direction along the central axis with respect to the insertion portion.

The first end may be fixed to the distal end of the insertion portion.

According to the present invention, treatment can be performed while suppressing interference of the indwelling objects existing in the surroundings.

### BRIEF DESCRIPTION OF THE DRAWINGS

FIG. 1 is a cross-sectional view schematically showing a configuration of a ligation device according to a first embodiment of the present invention.

FIG. 2 is a cross-sectional view showing a connecting portion between a clip unit and an applicator in the ligation device.

FIG. 3 is a diagram showing the appearance of the clip unit and flaps.

FIG. 4 is a diagram showing an example when the ligation device is used.

FIG. 5 is a diagram showing a proximal end portion of the clip unit in one process during use.

FIG. 6 is a diagram showing a proximal end portion of the clip unit in one process during use.

FIG. 7 is a diagram showing a proximal end portion of the clip unit in one process during use.

FIG. 8 is a diagram showing a modified example of the ligation device.

FIG. 9 is a diagram showing another example of the structure.

FIG. 10 is a diagram showing a distal end portion of a ligation device according to a second embodiment of the present invention.

FIG. 11 is a diagram showing an example when the ligation device is used.

FIG. 12 is a diagram showing a modified example of the ligation device.

FIG. 13 is a diagram showing a distal end portion of a ligation device according to a third embodiment of the present invention.

FIG. 14 is a diagram showing a distal end portion of a ligation device according to a modified example of the present invention.

FIG. 15 is a diagram showing a distal end portion of a ligation device according to a modified example of the present invention.

FIG. 16 is a diagram showing another example of a clip unit.

### DETAILED DESCRIPTION

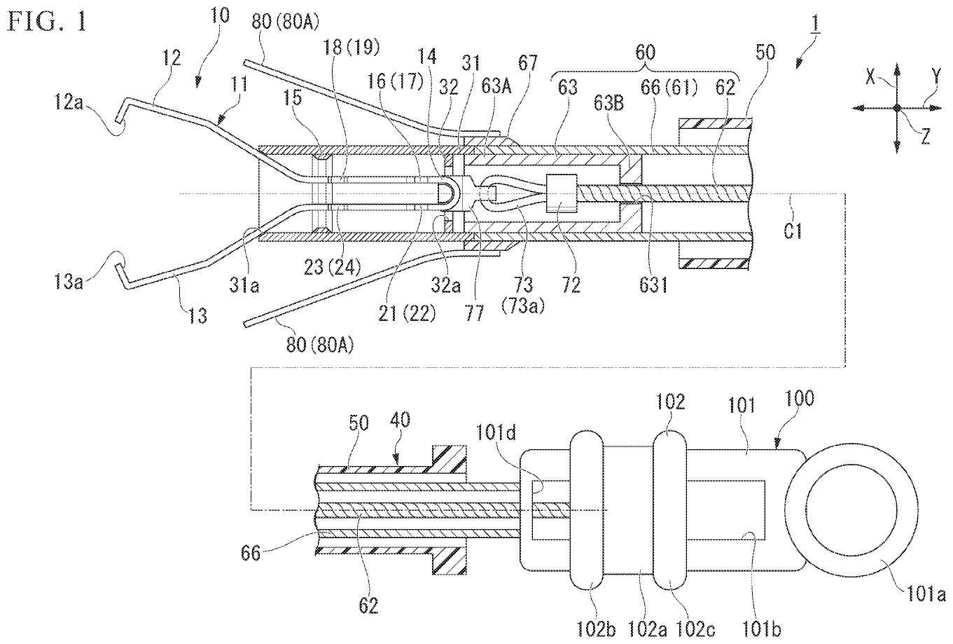

The first embodiment of the present invention will be described with reference to FIGS. 1 to 8.

FIG. 1 is a partial cross-sectional view of a ligation device 1, which is the treatment tool of the present embodiment. The ligation device 1 includes a clip unit (hereinafter, abbreviated as “clip”) 10 and an applicator 40. The clip 10 is removably connected to the distal end of the applicator 40. In the present embodiment, the clip 10 and the applicator 40 are connected by a connecting member 63.

As shown in FIG. 1, the clip 10 has an arm member (arm portion) 11 including a first arm 12 and a second arm 13. In the following description of the present embodiment, a direction in which the first arm 12 and the second arm 13 face each other is defined as a facing direction X, a direction parallel to the axis C1 of the holding tube 31 is defined as an axial direction Y, and a direction orthogonal to both the facing direction X and the axial direction Y is defined as an orthogonal direction Z. In the present embodiment, the axis C1 coincides with the central axis of the clip 10 and the insertion portion 60 of the applicator 40 described later. Therefore, in the following description, the axis C1 may be referred to as the “central axis C1”.

(Structure of Clip 10)

As shown in FIG. 1, the clip 10 includes an arm member 11 and a holding tube 31. The holding tube 31 is formed in a cylindrical shape and has an inner diameter through which the proximal end portion of the arm member 11 can enter. That is, the holding tube 31 is formed with a lumen into which the arm member 11 having the first arm 12 and the second arm 13 can enter.

(Structure of Arm Member 11)

The arm member 11 has a first arm 12, a second arm 13, and an intermediate portion 14. The first arm 12 and the second arm 13 extend from the proximal end side toward the distal end side and are arranged so as to face each other. The intermediate portion 14 is located between the proximal end of the first arm 12 and the proximal end of the second arm 13. In the side view shown in FIG. 1, the first arm 12 and the second arm 13 are formed at positions line-symmetric with respect to the axis C1.

The first arm 12 and the second arm 13 are naturally spaced from each other, and the distance between them increases as they approach the distal end. In the present specification, the “natural state” means a state in which an external force does not act on the arm member 11. For example, the state in which the first arm 12 and the second arm 13 do not receive a force from the inner peripheral surface of the holding tube 31 is a natural state. A claw 12a extending toward the second arm 13 side is formed at the distal end of the first arm 12. A claw 13a extending toward the first arm 12 side is formed at the distal end of the second arm 13.

The first arm 12 and the second arm 13 have an arcuate cross-sectional shape orthogonal to the longitudinal direction on the distal end side, and are formed into a rounded shape. By forming the first arm 12 and the second arm 13 in this shape, the strength against bending is improved, the frictional resistance with respect to the outer sheath 50 described later is reduced, and the first arm 12 and the second arm 13 can move forward and backward smoothly.

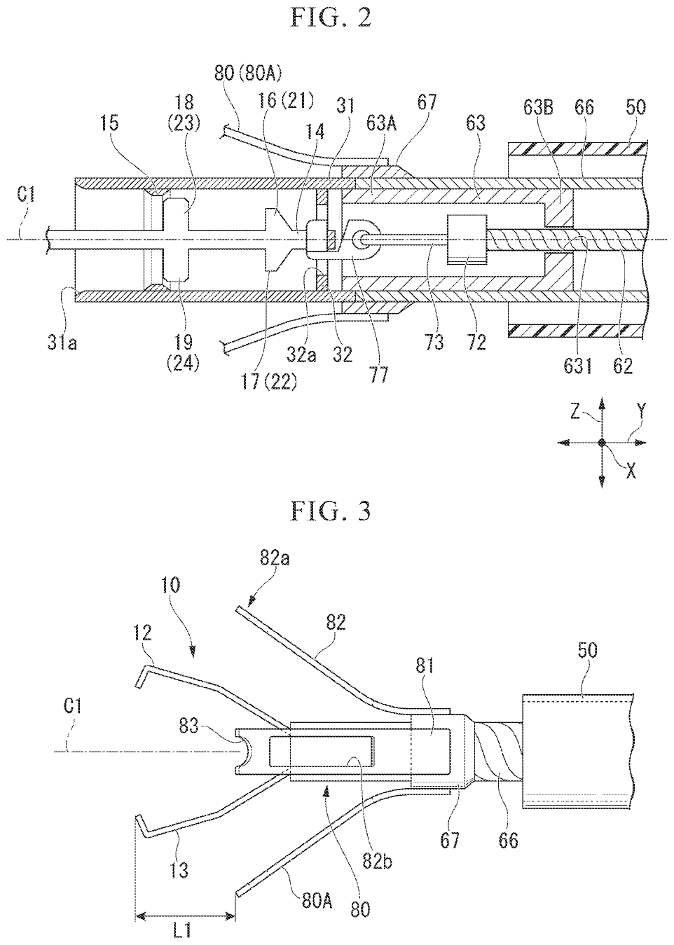

FIG. 2 is a cross-sectional view of the connecting portion between the clip 10 and the applicator 40 as viewed from a direction different from that of FIG. 1.

As shown in FIG. 2, two first locked portions 16 and 17 are provided at the proximal end portion of the first arm 12. The first locked portions 16 and 17 protrude from the first arm 12 in the Z direction. The first locked portions 16 and 17 protrude in opposite directions to each other.

In the plan view shown in FIG. 2, the first locked portion 16 and the first locked portion 17 are line-symmetric with respect to the axis C1. The proximal end surface of the first locked portion 16 is spaced from the central axis C1 toward the distal end side and is inclined with respect to the central axis C1. The distal end surface 161 of the first locked portion 16 is perpendicular to the axial direction Y. The proximal end surface 17a of the first locked portion 17 and the proximal end surface 16a of the first locked portion 16 are line-symmetric with respect to the axis C1. The distal end surface 17b of the first locked portion 17 and the distal end surface 16b of the first locked portion 16 are line-symmetric with respect to the axis C1.

In the first arm 12, two protrusions 18 and 19 are provided on the distal end side of the first locked portions 16 and 17. As shown in FIG. 2, the protrusions 18 and 19 protrude from the first arm 12 in the Z direction. The protrusion 18 and the protrusion 19 are line-symmetric with respect to the axis C1 in plan view. The length of the protrusions 18 and 19 protruding from the first arm 12 may be longer than the length of the first locked portions 16 and 17 protruding from the first arm 12 in the orthogonal direction Z.

The second arm 13 is provided with second locked portions 21, 22 and protrusions 23, 24 formed in the same manner as the first locked portions 16, 17 and the protrusions 18, 19 of the first arm 12. That is, the second locked portions 21 and 22 protrude in the Z direction. The protrusions 23 and 24 are provided on the distal end side of the second arm 13 with respect to the second locked portions 21 and 22, and protrude from the second arm 13 in the Z direction. The second locked portions 21 and 22 and the protrusions 23 and 24, and the first locked portions 16 and 17 and the protrusions 18 and 19, are arranged side by side in the facing direction X, respectively. In the plan view shown in FIG. 2, the second locked portions 21 and 22 overlap the first locked portions 16 and 17, respectively, and the protrusions 23 and 24 overlap the protrusions 18 and 19, respectively.

(Structure of Holding Tube 31)

On the inner peripheral surface of the holding tube 31, a stepped portion (engaging portion) 15 is provided so as to protrude inward of the holding tube 31 over the entire circumference. The inner diameter of the holding tube 31 is small at the position where the stepped portion 15 is provided. In the present embodiment, the position where the stepped portion 15 is provided on the inner peripheral surface along the longitudinal direction of the holding tube 31 is not particularly limited. For example, the stepped portion 15 may be provided on the inner peripheral surface on the distal end side of the holding tube 31.

In the present embodiment, the protrusions (engaged portions) 18, 19, 23, and 24 provided on the arm member 11 engage with the stepped portion 15 so as to be capable of restricting the arm member 11 from advancing with respect to the holding tube 31. The shape of the stepped portion 15 including the length protruding inward of the holding tube 31 is not particularly limited, but may be formed according to the shapes of the protrusions 18, 19, 23, and 24 of the arm member 11 described later, for example. Specifically, for example, slopes may be provided on the distal end side and the proximal end side of the stepped portion 15 along the longitudinal direction of the holding tube 31. Further, slopes may be provided at both ends of the protrusions 18, 19, 23, and 24 in the radial direction corresponding to the slopes provided on the stepped portion 15. By configuring the stepped portion 15 and the protrusions 18, 19, 23, and 24 according to the present embodiment in this way, when the arm member 11 moves forward and backward along the longitudinal direction of the holding tube 31, the protrusions 18, 19, 23, and 24 can easily move forward and backward while contacting the stepped portion 15.

On the inner peripheral surface of the holding tube 31 located closer to the proximal end side than the stepped portion 15, the locking portion 32 is formed so as to protrude in the direction toward the inside of the holding tube 31. In other words, the locking portion 32 is formed so as to protrude from the inner peripheral surface of the holding tube 31 in the direction toward the axis C1. In the present embodiment, in order to secure a space for press-fitting the connecting member 63 described later from the opening on the proximal end side of the holding tube 31, the locking portion 32 is formed at a predetermined distance on a more distal end side than the proximal end of the holding tube 31. That is, in the holding tube 31, the locking portion 32 is formed at a position between the proximal end of the holding tube 31 and the stepped portion 15.

The edge portion 32a on the axis C1 side of the locking portion 32 is formed in a circular shape coaxial with the holding tube 31. The portion of the first arm 12 on the proximal end side of the protrusions 18 and 19, the portion of the second arm 13 on the proximal end side of the protrusions 23 and 24, and the intermediate portion 14 can pass through the locking portion 32. Although the details will be described later, the first locked portions 16 and 17 formed on the first arm 12 and the second locked portions 21 and 22 formed on the second arm 13 pass over the locking portion 32 and come into contact with the locking portion 32 on the proximal end side of the locking portion 32, thereby being locked to the locking portion 32. In the present embodiment, the first locked portions 16 and 17 and the second locked portions 21 and 22 are locked to the locking portion 32, so that the movement of the arm member 11 toward the distal end side with respect to the holding tube 31 can be restricted.

Further, as shown in FIG. 1, a tapered surface 31a is formed on the inner peripheral surface of the distal end portion of the holding tube 31 over the entire circumference. The diameter of the tapered surface 31a increases toward the distal end side.

These members constituting the clip 10, including the arm member 11, are formed of a material such as a cobalt-chromium alloy, titanium, or stainless steel. The clip 10 is configured so that it can be observed under MRI (magnetic resonance imaging) fluoroscopy.

The arm member 11 is formed by punching, for example, a plate material made of a cobalt-chromium alloy or the like into a shape in which the first arm 12, second arm 13, intermediate portion 14, first locked portions 16, 17, second locked portions 21, 22, protrusions 18, 19, 23, 24 are developed in a plane. The arm member 11 bends the punched member at the connecting portion between the first arm 12 and the intermediate portion 14 and the connecting portion between the second arm 13 and the intermediate portion 14, so that the intermediate portion 14 and its surroundings are substantially C-shaped in side view and are integrally formed.

(Structure of Applicator 40)

Next, the configuration of the applicator 40 will be described.

As shown in FIG. 1, the applicator 40 has an outer sheath 50, an insertion portion 60, and an operation portion 100. The insertion portion 60 has a dimension capable of advancing and retracting in the outer sheath 50. The operation portion 100 is attached to the proximal end portion of the insertion portion 60.

The outer sheath 50 can be formed of, for example, a fluororesin such as PTFE (polytetrafluoroethylene) or a resin material such as HDPE (high-density polyethylene).

The insertion portion 60 includes a sheath portion 61, an operation wire (wire) 62, and a connecting member 63. The operation wire 62 is inserted through the sheath portion 61 so as to be capable of moving forward and backward. The connecting member 63 is provided to connect the holding tube 31 and the sheath portion 61.

In the present embodiment, the sheath portion 61 has a coil sheath 66 and a distal end member 67 fixed to the distal end portion of the coil sheath 66. The coil sheath 66 is formed of, for example, stainless steel such as SUS301 having high compressive strength. Specifically, as the coil sheath 66, a coil formed by tightly winding a wire (not shown) in the axial direction Y can be used. The coil sheath 66 has flexibility and is strong against a compressive force in the axial direction Y.

The distal end member 67 is formed of, for example, stainless steel in a cylindrical shape. The outer diameter of the distal end member 67 is larger than the outer diameter of the coil sheath 66 and the holding tube 31. The distal end member 67 and the coil sheath 66 are connected by laser welding or the like.

The operation wire 62 is formed of, for example, a single metal wire or a stranded wire. The distal end of the operation wire 62 is connected to the proximal end portion of the enlarged diameter portion 72. A loop portion 73 and a hook portion 77 are connected to the distal end portion of the enlarged diameter portion 72. In the present embodiment, the operation wire 62, the enlarged diameter portion 72, the loop portion 73, and the hook portion 77 are integrally configured and can move forward and backward together. Therefore, in the present embodiment, the enlarged diameter portion 72, the loop portion 73, and the hook portion 77 will be described as a part of the operation wire 66 as an extension of the operation wire 62.

The enlarged diameter portion 72 is formed in a cylindrical shape with, for example, metal. The outer diameter of the enlarged diameter portion 72 is smaller than the inner diameter of the coil sheath 66 and the connecting member 63. Further, the outer diameter of the enlarged diameter portion 72 is larger than the inner diameter of a through-hole 631 formed in the proximal end portion 63B of the connecting member 63. That is, the enlarged diameter portion 72 cannot pass through the through-hole 631.

The loop portion 73 is formed by folding back one wire 73a. The folded portion of the wire 73a is located on the distal end side, and both ends located on the proximal end side are fixed to the enlarged diameter portion 72 by brazing, resistance welding, or the like.

The hook portion 77 is connected to the distal end side of the loop portion 73. In the present embodiment, the hook portion 77 can connect the clip 10 and the applicator 40 by engaging with the intermediate portion 14 of the arm member 11. When the hook portion 77 rotates with respect to the loop portion 73, the engagement between the hook portion 77 and the central portion intermediate portion 14 is released. That is, the operation wire 62 is removably connected to the arm member 11.

As shown in FIGS. 1 and 2, the connecting member 63 is a tubular member having an outer diameter substantially equal to the inner diameter of the holding tube 31 and the inner diameter of the coil sheath 66. At the distal end portion 63A of the connecting member 63, a lumen is formed that has an inner diameter that allows the enlarged diameter portion 72, the loop portion 73, the hook portion 77, and the first arm 12 and the second arm 13 of the arm member 11 to enter. On the other hand, the inner diameter of the connecting member 63 is reduced in a part of the proximal end portion 63B. Specifically, the connecting member 63 is formed with the through-hole 631 at the proximal end portion 63B, which is smaller than the outer diameter of the enlarged diameter portion 72 and has an inner diameter equal to or larger than the outer diameter of the operation wire 62.

In the present embodiment, the material forming the connecting member 63 is not particularly limited, but for example, a material that can be elastically deformed can be used. When the connecting member 63 is configured as described above, the distal end portion 63A is press-fitted into the holding tube 31 from the proximal end side of the holding tube 31, and the proximal end portion 63B can be press-fitted into the coil sheath 66 from the distal end side of the coil sheath 66. In the present embodiment, the distal end portion 63A of the connecting member 63 is press-fitted into the holding tube 31, and is brought into close contact with the inner peripheral surface of the holding tube 31 located closer to the proximal end side than the locking portion 32, so that a frictional force is generated between the connecting member 63 and the holding tube 31. Similarly, the proximal end portion 63B of the connecting member 63 is press-fined into the distal end portion of the coil sheath 66 to bring them into close contact with each other, so that a frictional force is generated between the connecting member 63 and the coil sheath 66. The frictional force (static friction force) between the connecting member 63 and the holding tube 31 or the coil sheath 66 is determined by the static friction coefficient determined by the material forming the connecting member 63, the holding tube 31 and the coil sheath 66 and the degree of adhesion (that is, the force of pressing) between the connecting member 63 and the holding tube 31 or the coil sheath 66.

In the present embodiment, by the frictional force between the connecting member 63 and the holding tube 31 and the frictional force between the connecting member 63 and the coil sheath 66, the holding tube 31 and the coil sheath 66 are connected by the connecting member 63. In the natural state where no external force acts, the connection state between the holding tube 31 and the coil sheath 66 is not released. That is, in the endoscope clip 1 according to the present embodiment, the holding tube 31 and the coil sheath 66 are connected and integrated by the connecting member 63. The holding tube 31 and the coil sheath 66 are fixed by the connecting member 63, and do not move relative to the axis C1 direction of the holding tube 31. Therefore, when the operator pushes the slider 102 toward the distal end side to move the operation wire 62 toward the distal end side, the holding tube 31 does not move toward the distal end side.

In a state where the operation wire 62 and the enlarged diameter portion 72 connected to the distal end side of the operation wire 62 are pulled back to the proximal end side and the proximal end surface of the enlarged diameter portion 72 in contact with the proximal end portion 63B of the connecting member 63, when the force for pulling back the operation wire 62 becomes larger than the maximum static friction force between the connecting member 63 and the holding tube 31 or the coil sheath 66, the connecting member 63 can be pulled back to the proximal end side, in this state, the connecting member 63 can be moved to the proximal end side with respect to the holding tube 31 and the coil sheath 66.

At the distal end portion 63A of the connecting member 63, a lumen having an inner diameter larger than the outer diameter of the enlarged diameter portion 72, the loop portion 73, and the hook portion 77 is formed. Here, the outer diameters of the enlarged diameter portion 72, the loop portion 73, and the hook portion 77 mean the maximum dimensions in the radial direction orthogonal to the central axis C1 in these configurations. In the present embodiment, the hook portion 77 cannot rotate with respect to the loop portion 73 from the state where the hook portion 77 is located on the distal end side of the loop portion 73 in the holding tube 31 and the connecting member 63. In other words, the holding tube 31 and the distal end portion 63A of the connecting member 63 restrict the relative movement of the arm member 11 and the hook portion 77 in the radial direction.

The above-mentioned “the hook portion 77 cannot rotate with respect to the loop portion 73” means that the hook portion 77 cannot rotate with respect to the loop portion 73 to the extent that the hook portion 77 and the intermediate portion 14 are disengaged. Therefore, “the hook portion 77 cannot rotate with respect to the loop portion 73” does not mean that “the hook portion 77 cannot rotate with respect to the loop portion 73”.

As shown in FIG. 1, the operation portion 100 has an operation portion main body (handle) 101 and a slider 102.

The operation portion main body 101 is attached to the proximal end portion of the coil sheath 66. The operation portion main body 101 is formed in a rod shape extending in the axial direction Y, and a finger hook portion 101a, is provided at the proximal end portion. A slit 101b extending in the axial direction Y is formed in the operation portion main body 101.

The operation portion main body 101 is inserted through the slider 102. The slider 102 can slide (forward and backward) in the axial direction Y with respect to the operation portion main body 101. The proximal end of the operation wire 62 is connected to the slider 102. When the slider 102 is moved forward or backward, the operation wire 62 moves forward or backward in the axial direction Y. By advancing and retracting the operation wire 62, the enlarged diameter portion 72, the loop portion 73, the hook portion 77, and the arm member 11 of the clip 10 provided on the distal end side of the operation wire 62 can be advanced and retracted. As a result, the pair of the first arm 12 and the second arm 13 of the arm member 11 can be opened and closed.

The slider 102 is formed in a cylindrical shape. A recess 102a is formed on the outer peripheral surface of the slider 102 over the entire circumference. The slider 102 is formed with a flange portion 102b, a recess 102a, and a flange portion 102c in this order from the distal end side to the proximal end side in the axial direction Y. The pair of collar portions 102b and 102c have an elliptical shape when viewed in the axial direction Y. As a result, the slider 102 can be easily gripped, and space can be saved when packing the operation portion 100 of the endoscope clip 1.

By engaging the slider 102 with the slit 101b of the operation portion main body 101, the movement range of the slider 102 in the axial direction Y with respect to the operation portion main body 101 is limited.

As shown in FIGS. 1 and 2, a flap 80 is attached to the outer peripheral surface of the distal end member 67.

FIG. 3 shows the appearance of the clip 10 and the flap 80. The four flaps 80 are made of metal and have a rectangular frame-like basic shape with some parts removed. Each flap 80 has a proximal end portion (first end) 81 fixed to the coil sheath 66 and an expansion portion 82 connected to the proximal end portion 81. The distal end (second end) portion 82a of the expansion portion 82 is located closer to the proximal end side than the distal end of the first arm 12 and the distal end of the second arm 13, and each has an arc-shaped notch (reception portion) 83. The expansion portion 82 has a through-hole 82b in a part on the proximal end side of the notch 82a.

The proximal end portion 81 is fixed to the outer peripheral surface of the distal end member 67 by welding or the like. The fixed positions of the flaps are arranged at equal intervals in the circumferential direction of the outer peripheral surfaces of the coil sheath 66 and the distal end member 67, that is, at intervals of about 90° in phase. Of the four flaps 80, the phases of the two auxiliary flaps 80A coincide with the first arm 12 and the second arm 13, so that the auxiliary flap 80A is located on a plane (first plane) including the first arm 12, the second arm 13, and the central axis C1. The other two flaps 80 are located on a plane (second plane) orthogonal to the first plane and substantially parallel to the central axis C1.

A bending habit is attached to the boundary portion between the proximal end portion 81 and the expansion portion 82. Therefore, the expansion portion 82 of each flap 80 is substantially parallel to the axis C1 along the inner surface of the outer sheath 50 inside the outer sheath 50, and as shown in FIG. 3, it opens in the direction away from the holding tube 31 as it approaches the distal end outside the outer sheath 50. As a result, the distance between the distal end portion 82a and the central axis C1 is longer than the distance between the proximal end portion 81 and the central axis.

The angle formed by the proximal end portion 81 and the expansion portion 82 outside the outer sheath 50 can be appropriately adjusted. Examples of the material for forming the flap 80 include shape memory alloys such as stainless steel, cobalt-chromium alloys, and nickel titanium alloys.

The operation when the ligation device 1 configured as described above is used will be described. The ligation device 1 is introduced into the body via the endoscopic channel. When inserting the ligation device 1 into the endoscope, the user accommodates the clip 10 and the flap 80 in the outer sheath 50. Inside the outer sheath 50, the distal end portion 82a of the flap 80 moves to a position (closer position) closer to the central axis C1 than the position (spaced position) when it is outside the outer sheath 50.

When the ligation device 1 is protruded from the channel opening at the distal end of the endoscope and the outer sheath 50 is retracted with respect to the coil sheath 66, the first arm 12 and the second arm 13 are first opened by the elastic restoring force of the arm member 11. Subsequently, each flap 80 opens according to its bending habit by its own elastic restoring force.

The user moves the first arm 12 and the second arm 13 in the open state to positions sandwiching the target tissue. The first arm 12 and the second arm 13 are in a half-open state in which the target tissue of an average size can be ligated in a state where the protrusions 18, 19, 23, and 24 are in contact with the stepped portion 15. In a state where the target tissue is large, force is applied to the slider 102 to move it forward. As a result, the protrusions 18, 19, 23, 24 can pass over the stepped portion 15 and open the first arm 12 and the second arm 13 more widely.

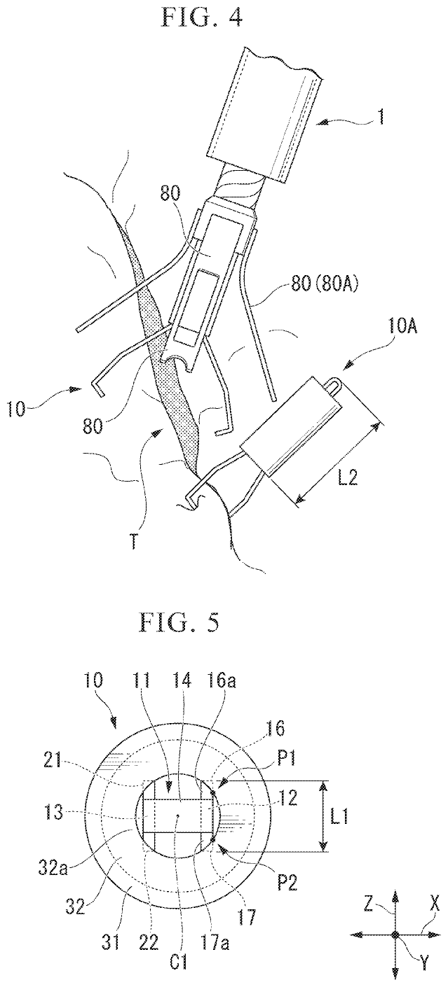

As shown in FIG. 4, when another clip unit (implant) 10A is already placed near the target tissue T, the clip unit 10A may interfere with the clip 10 due to falling toward the clip 10 or the like, which may interfere with the procedure. However, in the ligation device 1, since the flap 80 spreads around the clip 10, if the indwelling clip unit falls down, there is a high possibility that the flap 80 is first hit and supported. As a result, it is possible to preferably prevent the indwelling clip unit from interfering with the clip 10 and interfering with the procedure.

When it is determined that the tissue located between the first arm 12 and the second arm 13 may be ligated, the user retracts the slider 102 with respect to the operation portion main body 101. When the slider 102 retracts, the operation wire 62 is pulled, and the first arm 12 and the second arm 13 enter the holding tube 31 while sandwiching the tissue.

When the user grasps the operation portion main body 101 and pulls back the slider 102 further, the first locked portions 16 and 17 and the second locked portions 21 and 22 come into contact with the locking portion 32 of the holding tube 31, which is a contact state. In this process, first, as shown in FIG. 5, the proximal end of the first locked portion 16 on the proximal end surface 16a comes into point contact at the position P1 on the edge portion 32a of the holding tube 31, and the proximal end of the second locked portion 17 on the proximal end surface 17a makes point contact at the position P2 on the edge portion 32a, of the holding tube 31.

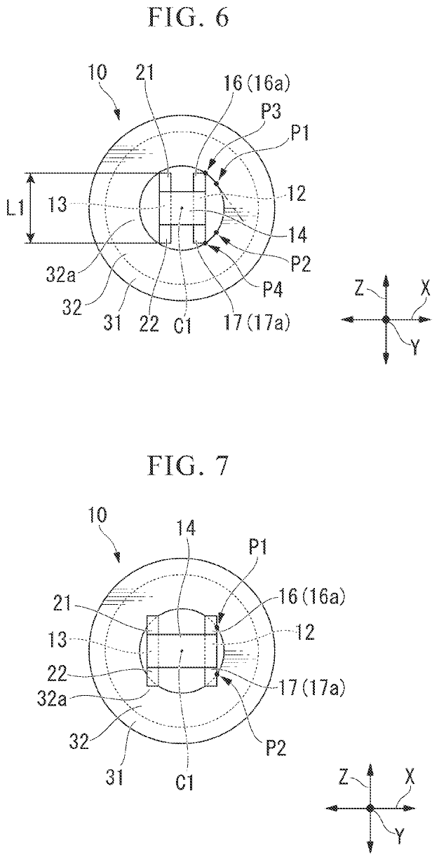

When the user further pulls back the slider 102 from the contact state, the arm member 11 is further moved to the proximal end side. At that time, the first arm 12 and the second arm 13 are elastically deformed in the direction approaching each other and pass through the locking portion 32. Specifically, the first locked portions 16 and 17 of the first arm 12 pass through the locking portion 32 in an elastically deformed state. At this time, the first locked portion 16 moves while making point contact with the locking portion 32, and the edge portion 32a of the locking portion 32 with which the first locked portion 16 makes contact changes from the position P1 to the position P3 shown in FIG. 6. At the same time, the first locked portion 17 moves while making point contact with the locking portion 32, and the edge portion 32a of the locking portion 32 with which the first locked portion 17 makes contact changes from the position P2 to the position P4. Further, both ends of the intermediate portion 14 are elastically deformed and approach the axis C1.

As a result of the above process, as shown in FIG. 6, both the distal end of the proximal end surface 16a of the first locked portion 16 and the distal end of the proximal end surface 17a of the first locked portion 17 come into contact with the edge 32a of the locking portion 32. The arm member 11 is in a riding state in which the first locked portions 16 and 17 and the second locked portions 21 and 22 pass over the locking portion 32. At this time, the first arm 12 and the second arm 13 of the arm portion 11 remain in the closed form. In the riding state, as shown in FIG. 6, the distance between the position P3 and the position P4 of the edge portion 32a is equal to the length L1 of the first locked portions 16 and 17.

When the user further pulls back the slider 102 from the riding state, the first locked portions 16, 17 and the second locked portions 21, 22 move beyond the locking portions 32 and further toward the proximal end side. Both the configuration on the distal end side of the first locked portions 16 and 17 in the first arm 12 and the configuration on the distal end side of the second locked portions 21 and 22 in the second arm 13 sequentially pass through the locking portion 32.

The first arm 12, the second arm 13, and the intermediate portion 14 that have passed through the locking portion 32 are not urged by the locking portion 32. Therefore, due to the elastic force of the intermediate portion 14, as shown in FIG. 7, the proximal end side of the first arm 12 and the proximal end side of the second arm 13 move in the facing direction X and are spaced from each other.

When the traction of the slider 102 is released, the distal end surfaces of the first locked portions 16 and 17 and the second locked portions 21 and 22 of the arm member 11 come into contact with the proximal end surface 32b of the locking portion 32 and are locked. As a result, in the arm member 11, the first arm 12 and the second arm 13 are closed, and the state in which the first arm 12 and the second arm 13 are tightly bound to the target tissue is maintained.

In parallel with the operation of the first locked portion and the second locked portion passing through the locking portion, the enlarged diameter portion 72 comes into contact with the proximal end portion 63B of the connecting member 63, and the connecting member 63 is retracted with respect to the holding tube 31 and the coil sheath 66. When the entire connecting member 63 moves out of the holding tube 31, the connection between the holding tube 31 and the sheath portion 61 is released. As a result, the hook portion 77 can rotate with respect to the loop portion 73. When the hook portion 77 rotates with respect to the loop portion 73, the connection between the arm member 11 and the operation wire 62 is released.

Finally, the clip 10 is spaced from the applicator 40 and placed in a ligated state of the target tissue T.

As described above, in the ligation device 1 of the present embodiment, during the indwelling procedure of the clip 10, in order for the flap 80 to suppress interference of various structures such as other implants that have been indwelled and tissues that are not the target of the procedure, for example, even in the case where a plurality of clip units are placed at narrow intervals to stop bleeding at a relatively large bleeding point, the procedure can be smoothly performed.

The flap 80 has a notch 82a at the distal end 82. When the size of the notch 82a is set to a size that allows the holding tube of the clip unit 10A to enter, the holding tube of the clip unit 10A that has fallen down can be suitably received. As a result, interference prevention performance is improved.

Further, since the flap 80 has a through-hole 82b, the front of the flap 80 can be visually recognized through the through-hole 82b in endoscopic observation. Therefore, the flap 80 is less likely to obstruct the view.

Further, the flap 80 has a bending habit at the boundary between the proximal end portion 81 and the distal end portion 82 and has an elastic restoring force. Therefore, it can be accommodated in the outer sheath 50 by simply protruding it from the outer sheath 50 to expand to a predetermined state and retracting it. The ligation device 1 has a configuration that allows the flap 80 to be easily brought into a desired state.

In the present embodiment, an example in which four flaps are provided has been described, but the number of flaps is not limited to this.

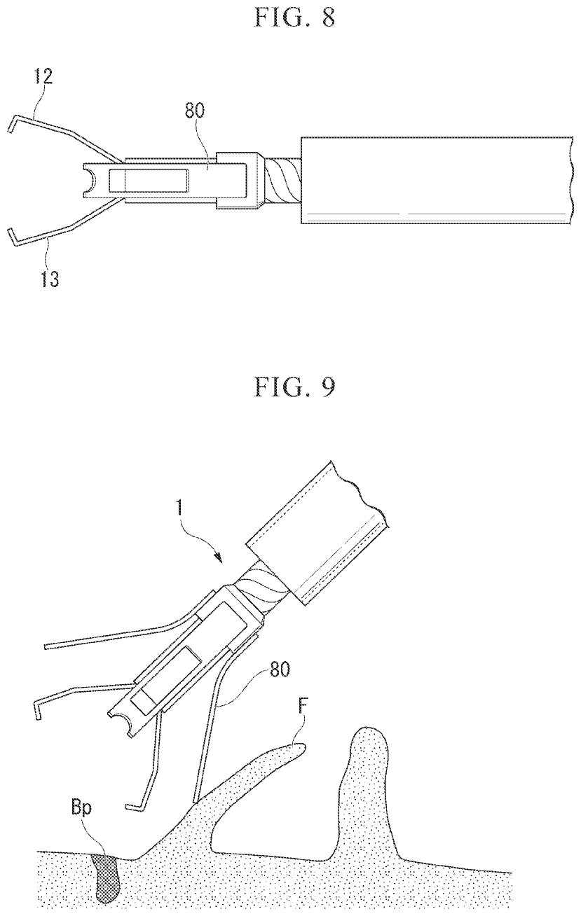

FIG. 8 shows a modified example of the configuration in which the auxiliary flaps are not provided and only the two flaps 80 are provided. In FIG. 8, one of the flaps 80 is hidden and invisible. In this modification, the two flaps 80 and the open first arm 12 and second arm 13 can be used to push away surrounding structures such as the indwelling clip unit.

In the case of this modification, if the phases of the first arm 12 and the second arm 13 and the phases of the two flaps are substantially the same, an interference suppression effect is reduced. Therefore, it is preferable that the ligation device have a structure in which the sheath and the arm member do not easily rotate relative to the axis C1. In the ligation device 1, since the pressing tube 31 and the inserting portion 60 are connected by the connecting member 63, the pressing tube 31 and the inserting portion 60 are unlikely to rotate relative to each other. Therefore, the configuration of the modified example can be suitably applied to the ligation device 1.

On the other hand, in the configuration of the first embodiment including four flaps, even if the phases of the first arm 12 and the second arm 13 and the phases of the two flaps are substantially the same, the remaining flaps sufficiently suppress interference. In order to obtain the effect, the holding tube 31 and the insertion portion 60 do not necessarily have to have a structure in which they are difficult to rotate relative to each other.

In the present embodiment, the positional relationship between the first arm 12, the second arm 13, and the flap 80 in the circumferential direction of the sheath portion can be appropriately set. The ligation device may be configured so that the arm member 11 can be rotated around the axis C1 with respect to the holding tube 31, and the positional relationship between the first arm 12 and the second arm 13 and the flap 80 may be changed.

In the flap 80, the presence/absence and shape of the notch 82a, and the presence/absence, shape, size and the like of the through-hole 82b may be appropriately set.

Further, the length of the flap 80 can also be appropriately set according to the dimensions of the structure for which interference is desired to be suppressed. For example, when the distance L1 (see FIG. 3) in the direction in which the central axis C1 extends between the tip of the first arm 12 or the second arm 13 and the distal end portion 82a of the flap at the spaced position is set to be equal to or less than the maximum length L2 of the implant in the indwelling state (the case of the clip unit 10A is shown in FIG. 4), the flap can be used to easily push or catch the structure.

The structure in this embodiment is not limited to an artificial object such as the implant described above. For example, living tissues such as folds F of the large intestine shown in FIG. 9 are also included. That is, interference between the clip 10 and the fold F can be suppressed by pressing the fold F using the flap 80. Thereby, for example, a procedure for stopping bleeding at the bleeding point Bp located behind the fold F can be easily performed.

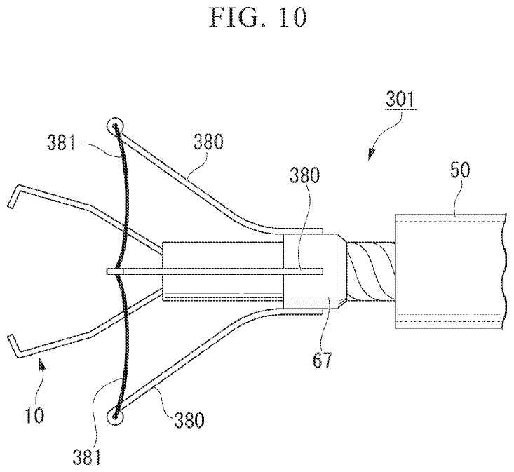

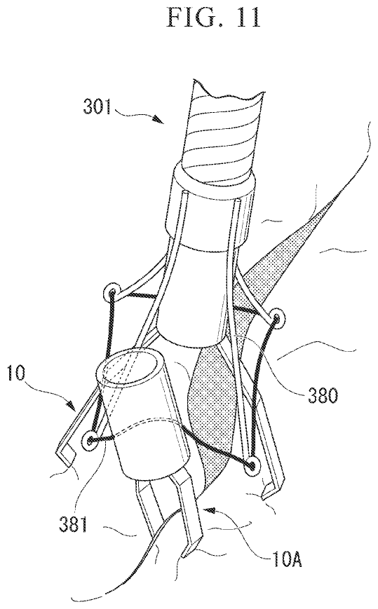

A second embodiment of the present invention will be described with reference to FIGS. 10 to 12. In the following description, the same reference numerals will be given to the configurations common to those already described, and duplicate description will be omitted.

FIG. 10 shows the distal end of the ligation device 301 according to the present embodiment. A rod-shaped flap 380 is attached to the distal end member 67 in a positional relationship substantially similar to that of the flap 80.

As the flap 380, a wire or the like formed of the same material as the flap 80 can be used. A thread 381 is attached to the distal end of each flap 380, and the thread 380 is stretched between the distal ends of adjacent flaps 380. The material of the thread 380 is not particularly limited as long as it has enough flexibility to be folded in half in the outer sheath 50.

FIG. 11 shows an example of the usage state of the ligation device 301. Unlike the first embodiment, the flap 380 does not have a through-hole, but is sufficiently thin as to not obstruct the view of the endoscope. The thread 381 arranged between the flaps 380 deforms according to the outer shape of the clip unit 10A or the like in contact with the flap 380, and preferably receives or pushes away the surrounding structure to prevent it from interfering with the clip unit 10 being indwelled.

When the flap 380 and the thread 381 are pulled into the outer sheath 50, the flap 380 becomes substantially linear and the thread 381 is folded so that the flap 380 and the thread 381 are smoothly housed in the outer sheath 50.

Similar to the ligation device 1 of the first embodiment, the ligation device 301 of the present embodiment can smoothly perform the procedure even when a plurality of clip units are placed at narrow intervals.

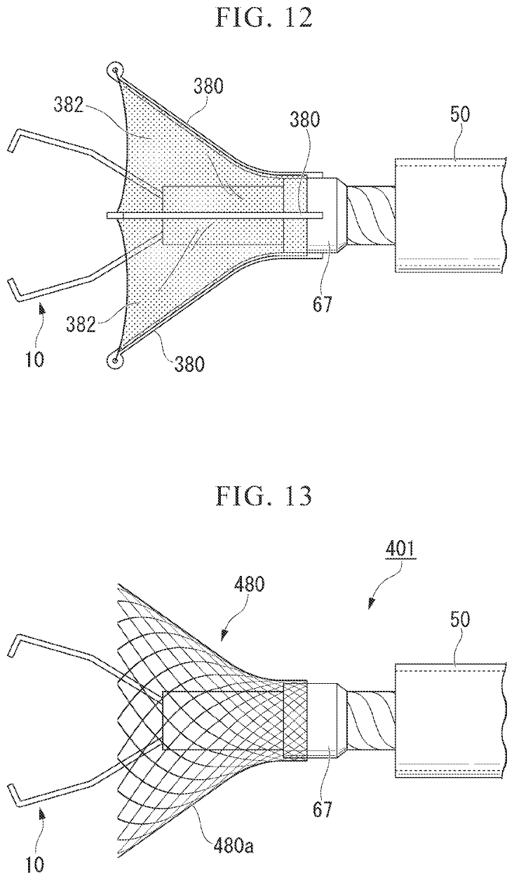

FIG. 12 shows a modified example of this embodiment. In this modification, instead of the thread 381, a transparent flexible film 382 is arranged between the adjacent flaps 380. As the film 382, a film made of various resins, cellophane, or the like can be used. In this modification, the film 382 suppresses interference with surrounding structures. Since the film 382 has transparency, the front of the film 382 can be confirmed endoscopically through the film 382.

Creases may be formed in the film 382 for the purpose of being smoothly accommodated in the outer sheath 50.

In the present embodiment, the attachment positions and dimensions of the thread 381 and the film 382 can be appropriately set. For example, the thread 381 may not be attached to the distal end of the flap 380, or may be a wide strip-shaped member. Further, the film 382 does not necessarily have to be arranged in the entire flap 380 space, and may be arranged in a part of the flap 380 space.

Also in this embodiment, the number of flaps and the arrangement mode can be appropriately set. Further, the thread or the film does not have to be arranged between some flaps.

A third embodiment of the present invention will be described with reference to FIG. 13.

FIG. 13 shows the distal end of the ligation device 401 according to the present embodiment. A net-like flap 480 is attached to the distal end member 67. The flap 480 is formed by knitting a plurality of strands 480a and has a self-expansion property. Such flaps 480 can be formed in a manner similar to known self-expanding stents. Examples of the material of the wire are metal, resin, and the like.

Outside the outer sheath 50, the flap 480 expands to a shape that covers the periphery of the clip 10 as shown in FIG. 13 due to self-expansion. The front of the flap 480 can be observed endoscopically through the mesh of the flap 480. When the flap 480 is pulled into the outer sheath 50, the distance between the strands 480a is shortened to reduce the diameter, and the flap 480 is preferably accommodated in the outer sheath 50.

Similar to the ligation device of the first embodiment and the second embodiment, the ligation device 401 of the present embodiment can smoothly perform the procedure even when a plurality of clip units are placed at narrow intervals.

In the present embodiment, the flap 480 does not necessarily have to be arranged over the entire circumference of the distal end member 67, and may be arranged at a part in the circumferential direction.

Although each embodiment of the present invention has been described above, the technical scope of the present invention is not limited to the above-described embodiment, and it is possible to make various changes or delete some component without departing from the spirit of the present invention.

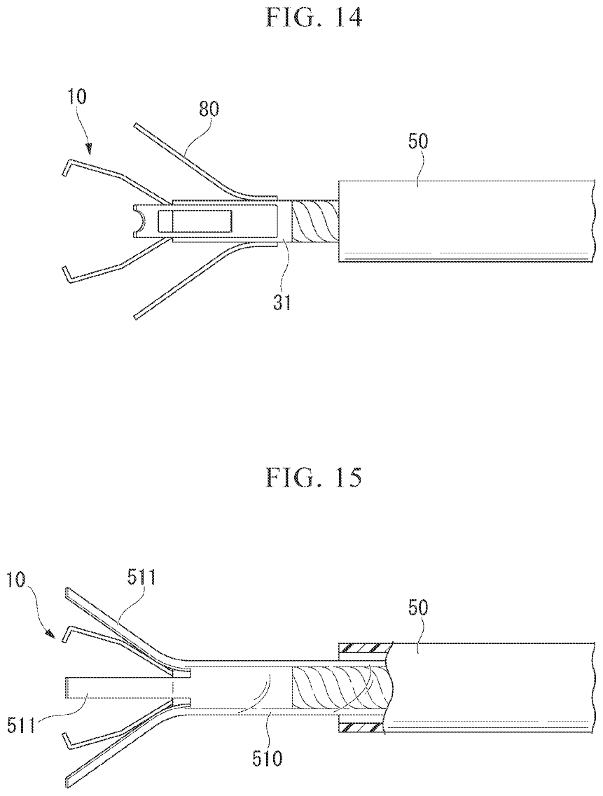

For example, as in the modified example shown in FIG. 14, the flap 80 may be attached to the holding tube 31. Even in such a configuration, the flap can be easily stored and expanded by moving the clip 10 in and out of the outer sheath 50.

The clip unit 101 including the flap 80, the holding tube 31, the first arm 12 and the second arm 13 can be applied to a reload unit for reloading the clip unit into the applicator after indwelling. By preparing a plurality of reload units and repeating the placement and reloading of the clip units, a plurality of clip units can be efficiently placed in the target tissue.

Any of the flaps of each of the above embodiments can be applied to the reload unit.

In the modified example shown in FIG. 15, the second sheath 510 is passed through the outer sheath 50, and the sheath portion (not shown) and the clip 10 are passed through the second sheath 510. The flap 511 is attached to the distal end of the second sheath 510.

In this modification, the flap 511 can be moved relative to the clip unit 10 by operating the second sheath 510. Therefore, the position of the flap 511 with respect to the clip 10 can be adjusted, and the flap 511 can be expanded and retracted independently of the clip 10.

When the second sheath 510 is formed of a transparent resin, the flap 511 may be integrally molded with the same material.

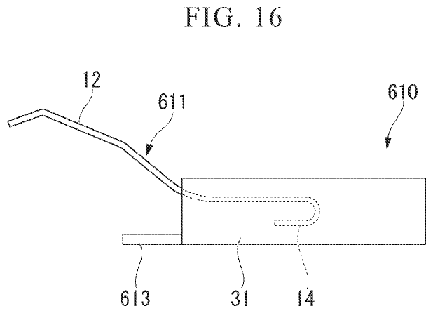

The clip unit of the present invention is not limited to one having the above-mentioned configuration. For example, the clip unit 610 shown in FIG. 16 may be used. The arm member 611 of the clip unit 610 includes only the first arm 12, and the second arm 613 is fixed to the holding tube 31. When the operation wire is operated to pull the arm member 611 into the holding tube 31, the first arm 12 approaches the second arm 613, and the tissue can be sandwiched between the first arm 12 and the second aim 613.

When applying the present invention to the clip unit 610, flaps may be provided on any of the sheath portion, the holding tube, and the second sheath.

The clip unit 610 is suitable for ligating tissues in a relatively narrow luminal organ such as the large intestine in a state where the wall surface of the organ and the holding tube 31 are substantially parallel to each other. Therefore, if the flap is provided on the plane including the first arm 12 and the second arm 613, it may be difficult to perform the treatment or it may be difficult to suppress interference of the surrounding structures. Therefore, when the present invention is applied to the clip unit 610, it is preferable to provide flaps at two positions facing each other with the axis C1 of the holding tube on the plane intersecting the plane including the first arm 12 and the second arm 613.

In the present invention, the urging means for moving the second end of the flap from the closer position to the separating position is not limited to the elastic restoring force of the flap itself. For example, a hinge may be provided at the boundary between the proximal end portion and the expansion portion, and an elastic body such as a spring may be arranged on the hinge to urge the hinge.

In each of the above embodiments and modifications, only one flap may be provided.

The treatment tool of the present invention is not limited to one provided with a clip unit at the distal end. For example, it may have a grasping forceps structure having a first arm and a second arm at the distal end portion.

What is claimed is:

1. A treatment system, comprising:
an implant placed in a body;
an insertion portion that is inserted into the body; and
a flap having a first end and a second end located at a position closer to a distal end than the first end, the flap extending from the first end to the second end,
wherein the second end has a receiving portion through which the implant can enter, and can be arranged at a spaced position, the spaced position being a position more spaced from a central axis of the insertion portion than the first end, and
the flap support the implant by abutting against the implant placed in the body.

2. The treatment system according to claim 1, further comprising:
an operation wire separably connected to at least one of the first arm and the second arm; and
a tubular holding tube into which at least one of the first arm and the second arm is inserted.

3. The treatment system according to claim 1, wherein
the first end is fixed to the insertion portion at a position closer to a distal end of the first arm and a distal end of the second arm, and
the second end is located closer to the distal end of the first arm and the distal end of the second arm.

4. The treatment system according to claim 1, wherein
the first end is fixed to the insertion portion at a a more proximal end side position than a distal end of the first arm and a distal end of the second arm,
the second end is located closer to a proximal end side than the distal end of the first arm and the distal end of the second arm, and
in a state where the second end is arranged at the spaced position, a length from the distal end of the first arm to the second end in a direction along the central axis and a length from the distal end of the second arm to the second end in the direction along the central axis are shorter than a maximum length of the implant.

5. The treatment system according to claim 1, wherein the first end is configured to be movable in a direction along the central axis with respect to the first arm and the second arm.

6. An applicator to which a clip unit having a first arm and a second arm is separably connected, the applicator comprising:
an insertion portion configured to be inserted into a body; and
a plurality of flaps extending from a first end to a second end arranged on a more distal end side than the first end,
wherein the second end is arranged at a spaced position, the spaced position being a position more spaced from a central axis of the insertion portion than the first end, and
in a state where the clip unit and the applicator are connected, the spaced position of the second end of at least one of the flaps is located on a second plane that intersects the first plane including the first arm, the second arm, and the central axis of the insertion portion.

7. The applicator according to claim 6, wherein the second end can be moved to a closer position, the closer position being a position closer to the central axis than the spaced position.

8. The applicator according to claim 7, further comprising:
an outer sheath having a tubular shape into which the insertion portion and the flap can be inserted,
wherein the second end is urged in a direction away from the central axis, and the second end moves to the spaced position by moving from an inside of the outer sheath to an outside.

9. The applicator according to claim 6, wherein the first end is configured to be movable in a direction along the central axis with respect to the insertion portion.

10. The applicator according to claim 6, wherein the first end is fixed to the distal end of the insertion portion.
